SEQUENCE LISTING (23 sequences)

SEQ ID NO: 1 - protein, 3 aa, Artificial Sequence
  Synthetic peptide
QAW

SEQ ID NO: 2 - protein, 3 aa, Artificial Sequence
  Synthetic peptide
QAW

SEQ ID NO: 3 - protein, 4 aa, Artificial Sequence
  Synthetic peptide
FQAW

SEQ ID NO: 4 - protein, 4 aa, Artificial Sequence
  Synthetic peptide
FQAW

SEQ ID NO: 5 - protein, 6 aa, Artificial Sequence
  Synthetic peptide
FLKQAW

SEQ ID NO: 6 - protein, 6 aa, Artificial Sequence
  Synthetic peptide
FLKQAW

SEQ ID NO: 7 - protein, 11 aa, Artificial Sequence
  Synthetic peptide
AMVSEFLKQAW

SEQ ID NO: 8 - protein, 11 aa, Artificial Sequence
  Synthetic peptide
AMVSEFLKQAW

SEQ ID NO: 9 - protein, 3 aa, Artificial Sequence
  Synthetic peptide
XXX

SEQ ID NO: 10 - protein, 6 aa, Artificial Sequence
  Synthetic peptide
XXXXXX

SEQ ID NO: 11 - protein, 3 aa, Artificial Sequence
  Synthetic peptide
QAW

SEQ ID NO: 12 - DNA, 18 bp, Artificial Sequence
  Synthetic primer
aggctgccccgactacgt

SEQ ID NO: 13 - DNA, 27 bp, Artificial Sequence
  Synthetic primer
gactttctcctggtatgagatagcaaa

SEQ ID NO: 14 - DNA, 19 bp, Artificial Sequence
  Synthetic primer
cagcaacagcaaggcgaaa

SEQ ID NO: 15 - DNA, 21 bp, Artificial Sequence
  Synthetic primer
ctggacctgtgggttgttgac

SEQ ID NO: 16 - DNA, 20 bp, Artificial Sequence
  Synthetic primer
tcgctcagggtcacaagaaa

SEQ ID NO: 17 - DNA, 22 bp, Artificial Sequence
  Synthetic primer
catcagaggcaaggaggaaaac

SEQ ID NO: 18 - DNA, 25 bp, Artificial Sequence
  Synthetic primer
acaagtcggaggcttaattacacat

SEQ ID NO: 19 - DNA, 25 bp, Artificial Sequence
  Synthetic primer
atgtgtaattaagcctccgacttgt

SEQ ID NO: 20 - DNA, 23 bp, Artificial Sequence
  Synthetic primer
atgctgcctgctcttactgactg

SEQ ID NO: 21 - DNA, 22 bp, Artificial Sequence
  Synthetic primer
ttgccattgcacaactcttttc

SEQ ID NO: 22 - DNA, 21 bp, Artificial Sequence
  Synthetic primer
agattactgctctggctccta

SEQ ID NO: 23 - DNA, 20 bp, Artificial Sequence
  Synthetic primer
caaagaaagggtgtaaaacg

(19) United States
(12) Patent Application Publication
(10) Pub. No.: US 2013/0261066 A1
(43) Pub. Date: Oct. 3, 2013

(54) TREATMENT OF INFLAMMATORY BOWEL DISEASES USING A TRIPEPTIDE

(71) Applicants: Ting Nie, Selden, NY (US); Basil Rigas, Old Field, NY (US); Nengtai Ouyang, Setauket, NY (US)

(72) Inventors: Ting Nie, Selden, NY (US); Basil Rigas, Old Field, NY (US); Nengtai Ouyang, Setauket, NY (US)

(73) Assignee: THE RESEARCH FOUNDATION OF STATE UNIVERSITY OF NEW YORK, Albany, NY (US)

(21) Appl. No.: 13/820,051

(22) PCT Filed: Aug. 31, 2011
(86) PCT No.: PCT/US11/50065

§ 371 Date: Jun. 4, 2013

Related U.S. Application Data
(60) Provisional application No. 61/378,708, filed on Aug. 31, 2010.

(51) Int. Cl. C07K 5/083; C07K 7/06; C07K 7/64

(52) U.S. Cl. CPC C07K 5/081; C07K 7/64; C07K 7/06
USPC 514/21.1; 530/321; 514/21.9; 514/21.6; 530/331; 530/329

(57) ABSTRACT

The present invention provides peptides and peptide conjugates for treating inflammatory bowel diseases, including ulcerative colitis and Crohn's disease. The peptides are derived from annexin-1, can be acetylated or conjugated to fatty acids, may be linear or cyclic, and may comprise D amino acids. Pharmaceutical compositions and methods of use are also disclosed.

### CROSS-REFERENCE TO RELATED APPLICATIONS

This application claims priority to U.S. Application No. 61/378,708 filed Aug. 31, 2010, which is incorporated herein by reference in its entirety. This application is related to PCT/US2008/058759, filed Mar. 28, 2008, which is incorporated herein by reference in its entirety.

### FIELD OF THE INVENTION

The present invention provides methods and compositions for treating inflammatory bowel diseases, including ulcerative colitis and Crohn's disease.

### BACKGROUND OF THE INVENTION

Inflammatory Bowel Diseases (IBDs) are common and increasing in prevalence. IBDs are a set of complex, life-long and for many patients devastating diseases, the etiologies of which are not completely understood, and for which there are no satisfactory treatments. The two major, clinically distinct forms of IBD are ulcerative colitis (UC) and Crohn's disease, which differ in the location and nature of the associated inflammation. Ulcerative colitis is characterized by ulcerations limited to the large intestine, colon and rectum. Crohn's disease commonly affects the terminal ileum of the small intestine and parts of the large intestine. Less common forms of inflammatory colitis include collagenous colitis, lymphocytic colitis, ischaemic colitis, diversion colitis, Behçet's syndrome, and indeterminate colitis.

Inflammatory bowel disease is also a substantial risk condition for colorectal cancer (CRC). The risk of CRC is primarily related to chronic inflammation and increases with the duration and extent of the inflammation. Further, earlier onset of symptoms is correlated with increased risk of malignancy and drugs that reduce inflammation are associated with reduced risk of CRC.

Treatment of IBD has been based on anti-inflammatory medications, with steroids having been the mainstay of treatment for years. The primary limitations of these treatments are variable efficacy and their side effects which, in the case of steroids, can limit dosing or duration of treatment or can force physicians to altogether discontinue them. Newer biological agents have also similar limitations, as well as high cost. It is clear that there is a pressing need for new agents for the control of the clinical manifestations of IBD.

### SUMMARY OF THE INVENTION

The invention provides annexin-1 peptides and conjugates of such peptides, including tripeptides, based on the structure of annexin A1 (“ANXA1”). The peptides suppress NF-κB activation and block inflammation associated with an inflammatory bowel disease. The invention further provides annexin A1-based peptides that are modified for increased efficacy. In certain embodiments, the peptides are conjugated for increased lipophilicity, increased drug availability in the blood, and/or increased stability against enzyme degradation. In an embodiment of the invention, an annexin-1 peptide is conjugated to a saturated or unsaturated fatty acid, including but not limited to stearic acid, oleic acid, linoleic acid, and other oleochemicals such as fatty acid methyl esters, fatty alcohols, fatty amines and glycerols, and intermediate chemical substances produced from oleochemicals, including alcohol ethoxylates, alcohol sulfates, alcohol ether sulfates, quaternary ammonium salts, monoacylglycerols, diacylglycerols, structured triacylglycerols and sugar esters. In an embodiment of the invention, the annexin-1 peptide is conjugated to stearic acid. In an embodiment of the invention, the annexin-1 peptide is acetylated. According to the invention, an annexin-1 peptide contains the amino acid sequence Gln-Ala-Trp. The size of the peptide is from 3 to about 50 amino acids. More preferably, the peptide is from 3 to about 20 amino acids or from 3 to about 10 amino acids. In one embodiment, the peptide is a tripeptide. The invention further provides pharmaceutical compositions of such peptides.

The effectiveness of annexin-1 peptides in preventing or reversing ulcerative colitis and Crohn's disease is demonstrated herein using the dextran sodium sulfate (DSS) and TNBS mouse models. The peptides reverse, in a dose-dependent manner, the inflammatory reaction of the colon and prevent the development of ulcerations. Further, no apparent side effects have been observed from the administration of annexin-1 tripeptides to mice.

Thus, the invention also provides a method of blocking inflammation associated with an inflammatory bowel disease (IBD). In one embodiment, the IBD is ulcerative colitis. In another embodiment, the IBD is Crohn's disease. In another embodiment, the IBD is collagenous colitis, lymphocytic colitis, ischaemic colitis, diversion colitis, Behçet's syndrome, or indeterminate colitis.

The invention provides a method of treating or reducing an inflammatory bowel disease in a mammal in need thereof, which comprises administering to the mammal a therapeutically effective amount of an annexin-1 peptide or conjugate. The invention also provides a method of treating or reducing ulcerative colitis in a mammal in need thereof, which comprises administering to the mammal a therapeutically effective amount of an annexin-1 peptide or conjugate. The invention further provides a method of treating or reducing Crohn's disease in a mammal in need thereof, which comprises administering to the mammal a therapeutically effective amount of an annexin-1 peptide or conjugate. There are several animal models of ulcerative colitis and Crohn's disease. The invention applies to all such animal models. In an embodiment of the invention, an annexin-1 peptide is conjugated to a moiety that increases lipophilicity of the conjugate. In one such embodiment, the annexin-1 peptide is conjugated to a fatty acid, such as, but not limited to stearic acid, oleic acid, or linoleic acid. In an embodiment of the invention, the mammal is a human.

### DESCRIPTION OF THE FIGURES

FIG. 1. Effect of MC12 in DSS-treated mice. Mice received 2% DSS in drinking water followed by treatment for 8 days with vehicle or MC12 (SEQ ID NO:1) intraperitoneally (ip). MC12 and QW3 (SEQ ID NO:1) have the same amino acid backbone. A: Body weight during treatment, expressed as percentage of baseline (day 0). B: Colon length, one from each of the six treatment groups. Changes in colon length, evident in the photos are quantified in C. Values are mean±SEM. *, statistically significant difference from the DSS group.

FIG. 2. MC12 ameliorated DSS-induced colitis. Paraffin sections of colonic tissues were stained with hematoxylin & eosin and their histological score was determined as in Methods. A: Control—Normal colonic mucosa. DSS—Severe inflammation including infiltration by inflammatory cells, edema, loss of crypts and ulcerations are seen in a DSS-treated mouse. D+40QW3—40 μg per mouse MC12 administered ip significantly decreased DSS-induced colonic inflammation. D+80QW3—80 μg per mouse MC12 administered ip also decreased DSS-induced colonic inflammation. B: Histology of DSS induced colitis. C: The histological score of the various study groups. Values are mean±SEM. *, statistically significant difference from the DSS group. Statistical comparisons: a vs. b: p<0.01; a vs. c: p<0.01; a vs. d: p<0.01; a vs. e: p<0.01; b vs. d: p>0.05; c vs. e: p<0.05

FIG. 3. The effect of MC12 on inflammation. The activity of myeloperoxidase (MPO) (A) and phospholipase A₂ (cPLA₂) (B) was determined in the control and treatment groups (DSS alone; DSS with 40 μg MC12; DSS with 80 μg MC12). Values are mean±SEM. *, statistically significant difference from the DSS group.

FIG. 4. MC12 inhibits NF-κB activation induced by DSS in vivo. Colon tissue sections were immunohistochemically stained with anti-phospho-NF-κB p65 antibody. A. Normal colon mucosa showed a few NF-κB nuclear-positive cells; B. Colon mucosa from DSS-treated animal showed many NF-κB nuclear-positive cells, most of which are crypt epithelial cells; C & D. Colon mucosa from DSS+MC12, 40 and 80 μg ip, treated animals showed less NF-κB nuclear translocation; E. The intensity of NF-κB staining is shown.

FIG. 5. Effect of MC-12 in DSS-treated mice. Mice received 2% DSS in drinking water followed by treatment for 8 days with vehicle or MC-12 intraperitoneally (ip) (A) or orally (po) (B). A, B: Body weight during treatment, expressed as percentage of baseline (day 0). C: Colon length, one from each of the six treatment groups. Changes in colon length, evident in the photos are quantified in D. Values are mean±SEM. *, statistically significant difference from the DSS group.

FIG. 6. MC-12 ameliorated DSS-induced colitis. Paraffin sections of colonic tissues were stained with hematoxylin & eosin and their histological score was determined. A: Normal colonic mucosa. B: Severe inflammation including infiltration by inflammatory cells, edema, loss of crypts and ulcerations are seen in a DSS-treated mouse. C: 25 mg/kg MC-12 administered ip significantly decreased DSS-induced colonic inflammation. D: 25 mg/kg MC-12 administered orally also decreased DSS-induced colonic inflammation. E: The histological score of the various study groups. Values are mean±SEM. *, statistically significant difference from the DSS group. Statistical comparisons: a vs. b: p<0.01; a vs. c: p<0.01; a vs. d: p<0.01; a vs. e: p<0.01; b vs. d: p>0.05; c vs. e: p<0.05

FIG. 7. MC-12 suppresses TNBS-induced colitis in mice. 2.5% TNBS was instilled intra-colonially into SJL/J mice to induce acute colitis, followed by intraperitoneal treatment with MC-12 or vehicle. A: The body weight of mice during treatment, expressed as percentage of baseline (day 1). B: The length of the colon of mice in the three treatment groups. C: Normal colonic mucosa. D: Colonic mucosa from a TNBS-treated mouse, showing severe crypt loss and inflammatory cell infiltration. E: 25 mg/kg MC-12 bid for 2 d decreased the inflammatory cell infiltration and crypt loss. F: The histological score of the various treatment groups. All values are mean±SEM. *, statistically significant difference from the TNBS group; **, statistically significant difference from the normal control.

FIG. 8. The effect of MC-12 on inflammation. The activity of myeloperoxidase (MPO) (A) and phospholipase A₂ (cPLA₂) (B) was determined in the control and treatment groups (DSS alone; DSS with 5 mg/kg MC-12; DSS with 25 mg/kg MC-12). Values are mean±SEM. *, statistically significant difference from the DSS group. C: The mRNA levels (mean±SEM) of the indicated cytokines were determined. All levels were significantly increased by DSS compared to controls and significantly decreased by MC-12 at either dose, compared to the DSS group.

FIG. 9. MC12 inhibits NF-κB activation induced by DSS in vivo. Colon tissue sections were immunohistochemically stained with anti-phospho-NF-κB p65 antibody. A. Normal colon mucosa showed a few NF-κB nuclear-positive cells; B. Colon mucosa from DSS-treated animal showed many NF-κB nuclear-positive cells, most of which are crypt epithelial cells; C & D. Colon mucosa from DSS+MC12, 5 & 25 mg/kg, ip, treated animals showed less NF-κB nuclear translocation; E. The percentage of positive cells in all groups is shown. MC 12 at both doses of 5 and 25 mg/kg by both ip and po administration showed marked decrease of NF-κB nuclear translocation; F. DSS markedly increased NF-κB DNA binding activity in NCM460 cells as measured by EMSA. The treatment with MC 12 at both concentrations of 30 and 300 μM significantly blocked this effect.

FIG. 10. The structure of stearate-MC12 (StMC12; SEQ ID NO:11). Stearic acid was added at the N terminal of the tripeptide Gln-Ala-Trp. The addition of the stearic acid increased the lipophilicity of MC12.

FIG. 11 StMC12 (SEQ ID NO:11) reduces DSS-induced colitis in mice. A. Normal control colon tissue; B. DSS induced severe colitis with a dense inflammatory infiltrate and nearly total loss of crypts; C: StMC12 significantly reduced colitis, restoring it to near normal (only residual inflammatory cells are present); D: The histological score of the three groups (mean±SEM): StMC12 significantly reduced DSS-induced colitis.

### DETAILED DESCRIPTION

A key regulator of inflammation is NF-κB, a transcription factor that is normally sequestered in the cytoplasm. When activated, it translocates into the nucleus where it regulates the expression of a multitude of genes related to inflammation. The invention provides peptides which have potent anti-inflammatory properties. Using two models of colitis, the peptides of the invention are shown to inhibit or reverse inflammation associated with IBDs.

In an embodiment of the invention, the annexin-1 peptide is a fragment of annexin A1 and contains the amino acid sequence Gln-Ala-Trp (SEQ ID NO:1). Various sizes of annexin-1 fragments can be used according to the invention. The size of the peptide can be from 3 to about 50 amino acids, or from 3 to about 20 amino acids, or from 3 to about 10 amino acids. In an embodiment of the invention, the annexin-1 peptide is a tripeptide. The following are non-limiting examples of the invention: Ac-Gln-Ala-Trp (SEQ ID NO:2), Ac-Phe-Gln-Ala-Trp (SEQ ID NO:4), Ac-Phe-Leu-Lys-Gln-Ala-Trp (SEQ ID NO:6), Gln-Ala-Trp (SEQ ID NO:1), Phe-Gln-Ala-Trp (SEQ ID NO:3), Phe-Leu-Lys-Gln-Ala-Trp (SEQ ID NO:5), Ac-Ala-Met-Val-Ser-Glu-Phe-Leu-Lys-Gln-Ala-Trp (SEQ ID NO:8), Ala-Met-Val-Ser-Glu-Phe-Leu-Lys-Gln-Ala-Trp (SEQ ID NO:7), or other fragments of annexin A1, that inhibit NF-κB activity.

With respect to fragments of annexin A1, one skilled in the art would understand that useful annexin-1 peptides include fragments of annexin A1 consisting of or containing the amino acid sequence Gln-Ala-Trp (SEQ ID NO:1), as well as peptides that contain the amino acid sequence Gln-Ala-Trp (SEQ ID NO:1), but otherwise vary in sequence relative to annexin A1. For example, as in certain of the peptides above, sequence variation, such as substitutions, insertions, or deletions, can be introduced at positions other than the Gln-Ala-Trp sequence. Such homologous fragments have sufficient sequence similarity with annexin A1 so as to have similar effects on a cell, and consequently on inflammation, as annexin A1. With respect to variants, one skilled in the art would appreciate that conservative mutations would more likely preserve the ability of the annexin A1 fragment or homolog to reduce inflammation and/or inhibit NF-κB activity. One assessment of NF-κB inhibitory activity is the ability of the variant or homologue to associate with the NF-κB dimer, thus limiting its ability to bind to the KB binding site on DNA. For example, nucleotide substitutions leading to amino acid substitutions at “non-essential” amino acid residues can be made in the sequence. A “non-essential” amino acid residue is a residue that can be altered from the wild-type sequence of annexin A1 in an annexin-1 peptide without significantly altering the biological activity, whereas an “essential” amino acid residue is required for biological activity.

As indicated, the peptides may be unmodified or modified, for example, acetylated at the N-terminus. In certain embodiments, the peptides are modified by conjugation to moieties which increase lipophilicity. Such conjugates are particularly suited for use in the GI tract. Examples of such moieties include, but are not limited to, stearic acid, oleic acid, and linoleic acid. In certain embodiments of the invention, the peptide is amidated at the C-terminus. One non-limiting example of a peptide of the invention is the tripeptide st-Gln-Ala-Trp (SEQ ID NO:11) with a stearic acid moiety at the N-terminus (designated stMC12 elsewhere herein):

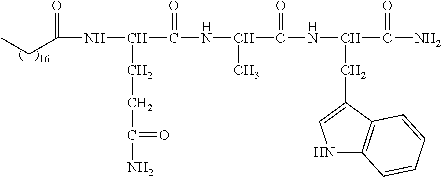

In an embodiment of the invention, the annexin-1 peptide contains D amino acids. Non-limiting examples include Ac-dGln-dAla-dTrp (SEQ ID NO:9), wherein, e.g., dGln represents D-glutamine), Ac-dPhe-dLeu-d-Lys-dGln-dAla-dTrp (SEQ ID NO:10). In certain embodiments, all of the amino acids are “D” amino acids. In other embodiments, the peptide contains D and L amino acids.

In certain embodiments of the invention, the annexin-1 peptides are cyclic. In one non-limiting embodiment, the cyclic peptide has head to tail cyclization. In another non-limiting embodiment, the cyclic peptide has side-chain to side-chain cyclization. Such side-chain to side-chain cyclization can involve formation of disulfide bonds between cysteine residues. Another side-chain cyclization involves amide bond formation between side chains. For example, suitably protected lysine and glutamic acid residues are assembled into a linear precursor, deprotected, and reacted. For a review of peptide cyclization methods, see, e.g., Davies, 2003, J. Peptide Sci. 9, 471-501, which is incorporated by reference. In certain embodiments, cyclic peptides of the invention incorporate L amino acids, D amino acids, or mixtures of both. In another embodiment, retro-inverso peptides are used. Retro-inverso peptides are made up of D-amino acids in a reversed sequence. The side chains assume a topology similar to that of the parent molecule made of L-amino acids, but with inverted amide peptide bonds. A non-limiting example of a cyclic annexin-1 peptide is Phe-Leu-Lys-Gln-Ala-Trp (SEQ ID NO:5), cyclized head to tail.

The amount of annexin-1 peptide or conjugate of the invention administered to a human is in the range from about 0.01 to about 50 mg/kg, or from about 0.02 to about 25 mg/kg, or from about 0.05 to about 2.5 mg/kg. Effective doses for administration to a human subject include, without limitation, 0.1, or 0.2, or 0.3, or 0.4, or 0.5, or 1.0, or 1.5, or 2.0 mg/kg body weight. Administration may be by injection, preferably subcutaneous or intramuscular. Total daily doses include without limitation, 5, 10, 15, 20, 25, 50, 75, 100, 150, or 200 mg. When administered orally, the dosages of the active peptides may be increased 2-10 fold or higher. The amounts may be administered in single or divided doses. The exact dose to be administered is determined by the attending clinician and may depend on the severity of inflammation, as well as upon the age, weight and condition of the individual. Administration should begin at the first sign of symptoms or shortly after diagnosis of the IBD.

Animal studies can be used in formulating a range of dosages for use in humans in accordance with the invention. The dosage of such agents lies preferably within a range of circulating concentrations that include the ED₅₀ with little or no toxicity. The dosage may vary within this range depending upon the dosage form employed and the route of administration utilized. For example, the amount of a peptide of the invention administered to a mouse is normally in the range from about 1 to about 25 mg/kg. Thus, useful daily doses administered to a mouse include, without limitation 50 μg, or 100 μg, or 300 μg, or 500 μg. For a 20 gm mouse, these amounts correspond to 2.5, 5, 15, or 25 mg/kg body weight. Levels in plasma may be measured, for example, by high-performance liquid chromatography.

One of skill in the art would understand that equivalent dosage amounts for humans vs. other mammals can be extrapolated, for example, by normalization to body surface area (BSA). See, e.g., Estimating the Maximum Safe Starting Dose in Initial Clinical Trials for Therapeutics in Adult Healthy Volunteers, 2002, Center for Drug Evaluation and Research, U.S. Department of Health and Human Services, Food and Drug Administration. The following table provides Kₘ factors for various animals and humans. To convert from an animal to a human, multiply the animal dose by its Kₘ factor and divide by the human Kₘ factor.

TABLE 1
Conversion of animal doses to human equivalent dose (HED) based on
BSA

| Species | Kₘ factor |
|---|---|
| Human (60 kg adult) | 37 |
| Human (20 kg child) | 25 |
| Mouse | 3 |
| Hamster | 5 |
| Rabbit | 12 |
| Dog | 20 |

As used herein, the term “effective amount” in the context of administering a therapy to a subject refers to the amount of a therapy which is sufficient to achieve one or more of the following effects: (i) reduce or ameliorate the severity of inflammation or a symptom associated therewith; (ii) reduce the duration of inflammation or a symptom associated therewith; (iii) prevent the progression of inflammation or a symptom associated therewith; (iv) cause regression of inflammation or a symptom associated therewith; (v) prevent the development or onset of inflammation or a symptom associated therewith; (vi) prevent the recurrence of inflammation or a symptom associated therewith; (vii) reduce hospitalization of a subject; (viii) reduce hospitalization length; and/or (ix) enhance or improve the prophylactic or therapeutic effect(s) of another therapy.

Pharmaceutical compositions may be prepared as medicaments to be administered by any route. Non-limiting examples are oral, parenteral (including subcutaneous, intramuscular, and intravenous), rectal, or transdermal administration. A suitable administration route may best be determined by a medical practitioner for each patient. Pharmaceutically acceptable carriers and their formulations are described in standard formulation treatises, e.g., Remington's Pharmaceutical Sciences by E. W. Martin.

In an embodiment of the invention, compounds of the invention are administered orally. For example, as exemplified herein, the compounds may be dissolved in corn oil or another such dietary substance and directly ingested. In an embodiment of the invention, an annexin-1 peptide or conjugate is dissolved in an acceptable carrier and encapsulated. In another embodiment, an annexin-1 peptide or conjugate is dissolved in an acceptable carrier and consumed directly, for example as a syrup or taken with or mixed into food. In a preferred embodiment, an annexin-1 peptide or conjugate of the invention is in a dosage form adapted for localized delivery to the large or small intestine, or both.

The compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a peptide preferably in purified form, together with a suitable amount of carrier so as to provide the form for proper administration to the patient. The formulation should suit the mode of administration. In a preferred embodiment, the compositions or single unit dosage forms are sterile and in suitable form for administration to a subject, preferably a mammalian subject, and more preferably a human subject.

Excipients such as diluents increase the bulk of a solid pharmaceutical composition, and may make a pharmaceutical dosage form containing the composition easier for the patient and care giver to handle. Diluents for solid compositions include, but are not limited to, microcrystalline cellulose (e.g., AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, or talc.

Solid pharmaceutical compositions that are compacted into a dosage form, such as a tablet, may include, but are not limited to, excipients whose functions include, but are not limited to, helping to bind the active ingredient and other excipients together after compression, such as binders. Binders for solid pharmaceutical compositions include, but are not limited to, acacia, alginic acid, carbomer (e.g., CARBOPOL®), carboxymethylcellulose sodium, dextrin, ethyl cellulose, gelatin, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, magnesium aluminum silicate, maltodextrin, methylcellulose, polymethacrylates, povidone (e.g., KOLLIDON®, PLASDONE®), pregelatinized starch, sodium alginate, or starch.

The dissolution rate of a compacted solid pharmaceutical composition in the patient's stomach may be increased by the addition of a disintegrant to the composition. Excipients which function as disintegrants include, but are not limited to, alginic acid, carboxymethylcellulose calcium, carboxymethylcellulose sodium (e.g., AC-DI-SOL®, PRIMELLOSE®), colloidal silicon dioxide, croscarmellose sodium, crospovidone (e.g., KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB®), or starch.

Glidants can be added to improve the flowability of a non-compacted solid composition and to improve the accuracy of dosing. Excipients that may function as glidants include, but are not limited to, colloidal silicon dioxide, magnesium trisilicate, powdered cellulose, starch, talc, or tribasic calcium phosphate.

When a dosage form such as a tablet is made by the compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease the release of the product from the die. Excipients that function as lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, or zinc stearate.

Flavoring agents and flavor enhancers make the dosage form more palatable to the patient. Common flavoring agents and flavor enhancers for pharmaceutical products that may be included in the composition of the invention include, but are not limited to, maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the invention, the active ingredient and any other solid excipients are suspended in a liquid carrier such as water, vegetable oil, alcohol, polyethylene glycol, propylene glycol, or glycerin. As used herein, “active ingredient” means annexin-1 peptides or conjugates of the invention.

Liquid pharmaceutical compositions may contain emulsifying agents to disperse uniformly throughout the composition an active ingredient or other excipient that is not soluble in the liquid carrier. Emulsifying agents that may be useful in liquid compositions of the invention include, but are not limited to, gelatin, egg yolk, casein, cholesterol, acacia, tragacanth, chondrus, pectin, methyl cellulose, carbomer, cetostearyl alcohol, or cetyl alcohol.

Liquid pharmaceutical compositions of the invention may also contain a viscosity enhancing agent to improve the mouth-feel of the product and/or coat the lining of the gastrointestinal tract. Such agents include, but are not limited to, acacia, alginic acid, bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, or xanthan gum.

Sweetening agents such as sorbitol, saccharin, sodium saccharin, sucrose, aspartame, fructose, mannitol, or invert sugar may be added to improve the taste.

Preservatives and chelating agents such as alcohol, sodium benzoate, butylated hydroxy toluene, butylated hydroxyanisole, or ethylenediamine tetraacetic acid may be added at levels safe for ingestion to improve storage stability.

According to the invention, a liquid composition may also contain a buffer such as gluconic acid, lactic acid, citric acid or acetic acid, sodium gluconate, sodium lactate, sodium citrate, or sodium acetate.

Generally, an effective amount of the agents described above will be determined by the age, weight and condition or severity of disease of the recipient. Dosing may be one or more times daily, or less frequently, and may be in conjunction with other compositions as described herein. It should be noted that the present invention is not limited to the dosages recited herein.

Peptides and conjugates of the invention may be coadministered with other therapies used for treatment of colitis. Such therapies include aminosalicylates, drugs that contain 5-aminosalicyclic acid (5-ASA), and help control inflammation. Sulfasalazine is a combination of sulfapyridine and 5-ASA. The sulfapyridine component carries the anti-inflammatory 5-ASA to the intestine. However, sulfapyridine may lead to side effects such as nausea, vomiting, heartburn, diarrhea, and headache. Other 5-ASA agents, such as olsalazine, mesalamine, and balsalazide, have a different carrier, fewer side effects, and may be used by people who cannot take sulfasalazine. 5-ASAs are given orally, through an enema, or in a suppository, depending on the location of the inflammation in the colon. Most people with mild or moderate ulcerative colitis are treated with this group of drugs first. This class of drugs is also used in cases of relapse.

The anti-inflammatory agent may be a corticosteroid (e.g., prednisone, methylprednisone, and hydrocortisone), a glucocorticosteroic, or dexamethasone. Such agents can be given orally, intravenously, through an enema, or in a suppository, depending on the location of the inflammation. These drugs can cause side effects such as weight gain, acne, facial hair, hypertension, diabetes, mood swings, bone mass loss, and an increased risk of infection. For this reason, they are not recommended for long-term use, although they are considered very effective when prescribed for short-term use.

Immunomodulators such as azathioprine and 6-mercapto-purine (6-MP) reduce inflammation by affecting the immune system. These drugs are used for patients who have not responded to 5-ASAs or corticosteroids or who are dependent on corticosteroids. Immunomodulators are administered orally, however, they are slow-acting and it may take up to 6 months before the full benefit. Patients taking these drugs are monitored for complications including pancreatitis, hepatitis, a reduced white blood cell count, and an increased risk of infection. Cyclosporine A may be used with 6-MP or azathioprine to treat active, severe ulcerative colitis in people who do not respond to intravenous corticosteroids.

The peptides of the invention may be coadministered with naturopathic agents. Such agents include, but are not limited to, supplements of omega 3/6 fish oils, probiotics, L-glutamine and N-Acetylglucosamine. The peptides can also be coadministered with diet related therapies. For example therapies for colitis frequently involves avoidance of certain foods (roughage, seeds, dairy, gluten, seedy fruits, broccoli, beets and meats).

The above-described administration schedules are provided for illustrative purposes only and should not be considered limiting. A person of ordinary skill in the art will readily understand that all doses are within the scope of the invention.

It is to be understood and expected that variations in the principles of invention herein disclosed may be made by one skilled in the art and it is intended that such modifications are to be included within the scope of the present invention.

Throughout this application, various publications are referenced. The disclosures of these publications in their entireties are hereby incorporated by reference into this application to more fully describe the state of the art to which this invention pertains. The following examples further illustrate the invention, but should not be construed to limit the scope of the invention in any way.

### EXAMPLES

### Example 1

### MC12 Reduces Experimental Colitis in Mice

Female C57BL/6 and SJL/J mice (Taconic, Hudson, N.Y.), 7-9 weeks old, were kept under controlled temperature (25° C.) with a 12/12-hour light-dark cycle and free access to standard diet and drinking water. The mice were allowed to acclimate for 7 days before the start of experiments.

The mice received 2% dextran sulfate sodium (DSS, MW 36,000 to 50,000, MP Biomedicals, Solon, Ohio) in drinking water for 7 days; control mice received regular drinking water. During the period when DSS was administered, treated mice were given MC12 40 or 80 μg/mouse intraperitoneally (i.p.) whereas the control group was given saline i.p. The mice were weighed and monitored for rectal bleeding or prolapse daily. All mice were euthanized at the end of the study. Blood samples were collected and colons were dissected and their length was measured. The middle part of colon was fixed in 4% neutralized formalin and the rest was frozen for molecular analyses.

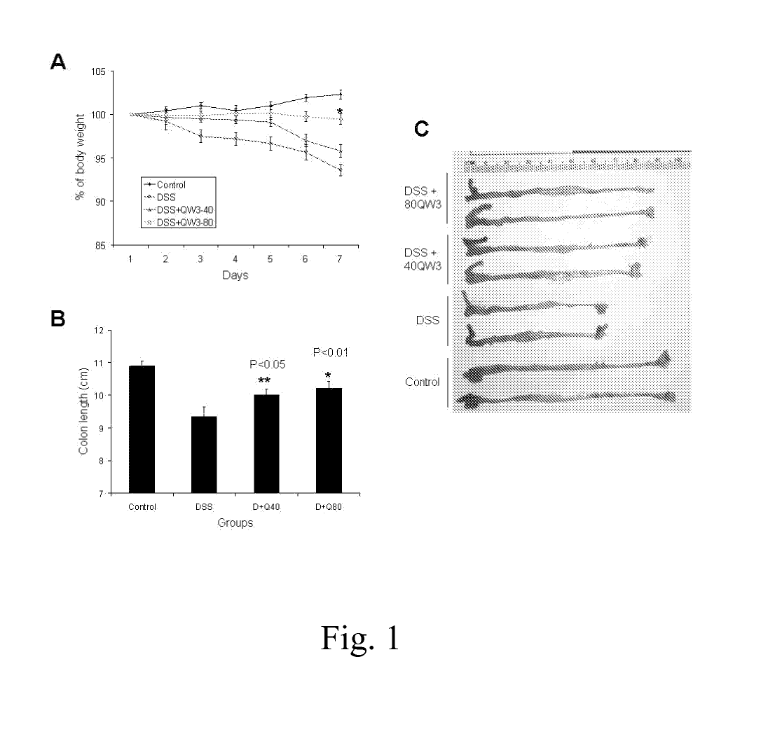

FIG. 1A shows changes of the body weight and the treatment groups, while untreated animals gained weight. MC12 (SEQ ID NO:1) is identified in FIG. 1 as QW3. Those receiving DSS lost on average 7% of their weight by day 7. In contrast, animals treated with MC 12 showed a dose-dependent reduction in weight loss, with those receiving the higher dose of MC12 (80 μg/day), showing essentially no change in their weight. FIG. 1 B and C show changes in the length of the colon of these mice. Compared to control, the colon of DSS-treated mice was shorter by 1.5 cm (p<0.01). Administration of MC12 prevented this reduction in colon length in a dose-dependent manner.

Paraffin-embedded tissue sections were stained with hematoxylin and eosin (H&E). In these sections, the histological score was determined based on epithelial denudation, loss of crypts, ulceration, edema and leukocyte infiltration using criteria that have been described previously (see Perretti, F. et al., 2009, Nat Rev Immunol 9:62-70)

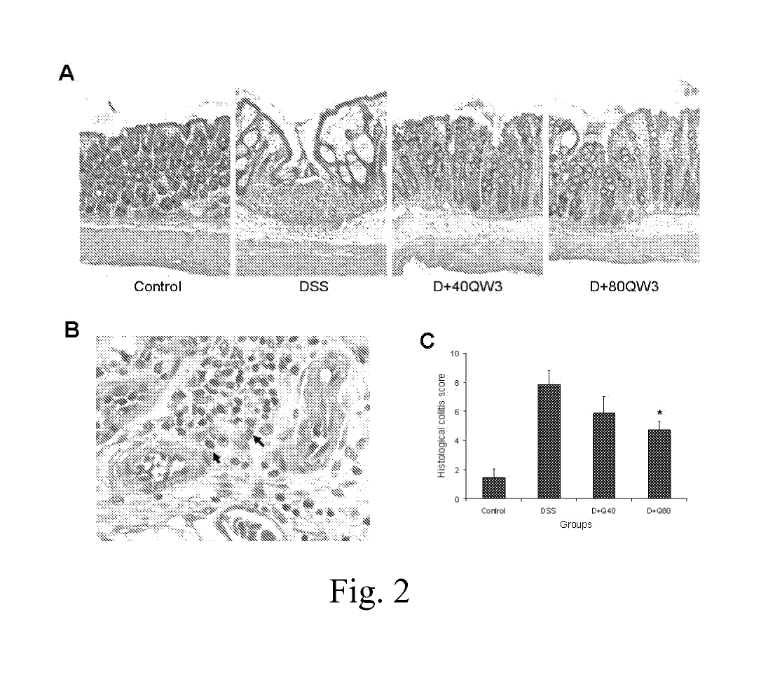

As expected, DSS induced colitis in these mice. As shown in FIG. 2, the histological colitis score was increased 5.5-fold by DSS, compared to control. MC12 (SEQ ID NO:1) reduced the histological colitis score dose-dependently, with this effect becoming statistically significant at the higher MC12 dose. The histological sections shown in FIG. 2 demonstrate the induction of inflammation, accumulation of mucus and the development of ulcers in the colonic mucosa. The granulocytes present in the mucosa establish the presence of acute inflammation. Treatment with MC12 reduced inflammation and the size of goblet cells and essentially restored the integrity of the mucosa.

### Example 2

### MC12 Reduces Inflammation in Murine Colonic Mucosa Induced by DSS

To further assess the effect of MC12 on the inflammatory changes associated with DSS induced colitis, myeloperoxidase (MPO) activity and cytosolic phospholipase A₂ (cPLA₂) in tissue samples was determined.

MPO activity is an indicator of the degree of acute inflammation in a given tissue. MPO activity was measured using a commercial kit and following the instructions of the manufacturer (Invitrogen, Eugene, Oreg.). Briefly, a portion of colon tissue was homogenized in PBS and centrifuged at 10,000×g for 15 min and 50 μl of supernatant from each sample were added into a 96-well microplate. 50 μl of 2×APF working solution was added to all sample and standard wells and the plate was incubated at room temperature for 30 min. The reaction was stopped by adding 10 μl of 10× chlorination inhibitor. The fluorescence intensity was measured using a Multiplate Reader (Molecular Devices) at excitation at 485 nm, emission at 530 nm.

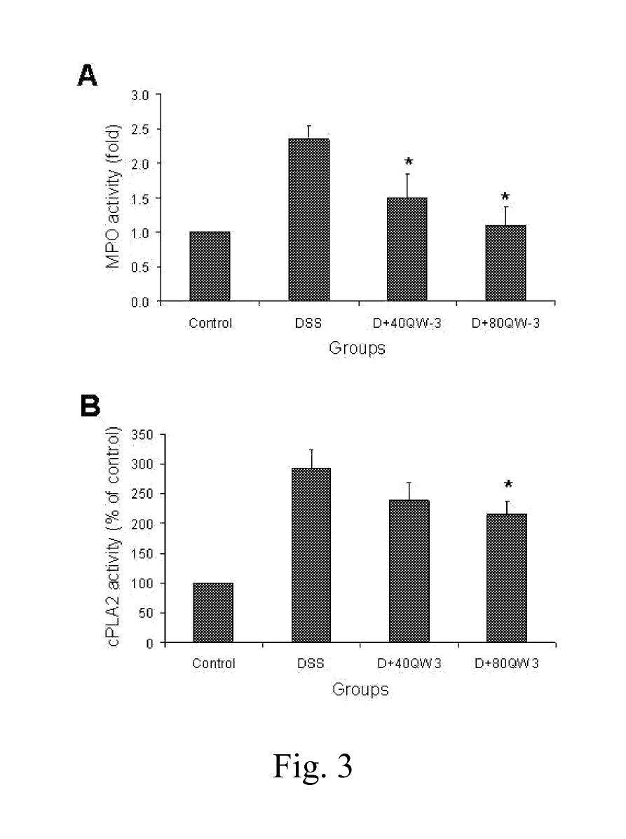

As shown in FIG. 3A, DSS increased MPO activity 2.4-fold compare to controls. Mice treated with MC12 had a significant (p<0.01) and concentration-dependent reduction in MPO activity, which was essentially normalized at the 80 μg dose.

cPLA₂ enzymes release fatty acids from the second carbon group of glycerol. This particular phospholipase specifically recognizes the sn-2 acyl bond of phospholipids and catalytically hydrolyzes the bond releasing a lysopospholipid and arachidonic acid; the latter is then modified by cyclooxygenases into active compounds called eicosanoids by cyclooxygenases. Eicosanoids include prostaglandins and leukotrienes which are categorized as inflammatory mediators. As shown in FIG. 3B, DSS increased cPLA₂ activity 2.9-fold compare to controls. Treatment with MC12 decreased cPLA₂ activity by 18% at 40 μg and 26% at the dose of 80 μg (p<0.05).

### Example 3

### MC12 Inhibits the Activation of NF-κB in Murine Colonic Mucosa

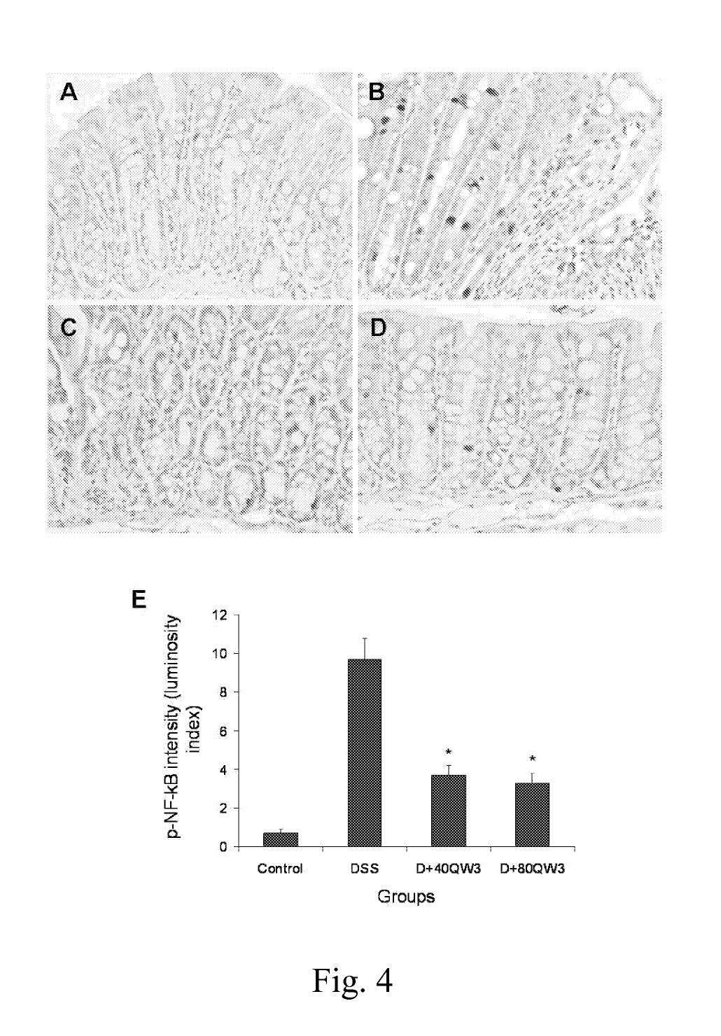

NF-κB is the master regulator of inflammation, controlling the transcription of many genes related to this complex process. It is also the molecular target of the activity of MC 12. Therefore, we determined by immunohistochemistry the level of NF-κB activation in the colonic mucosa of our four study groups of mice. There was minimum or known activation of NF-κB in the colon of control animals (FIG. 4A). DSS, as expected, activated NF-κB in both colonic crypts and stromal cells (FIG. 4B). MC12 markedly decreased this effect of DSS (FIGS. 4C, D and E).

### Example 4

### MC-12 Reduces Experimental Colitis in Mice

Female C57BL/6 and SJL/J mice (Taconic, Hudson, N.Y.), 7-9 weeks old, were kept under controlled temperature (25° C.) with a 12/12-hour light-dark cycle and free access to standard diet and drinking water. The mice were allowed to acclimate for 7 days before the start of experiments.

The mice received 2% dextran sulfate sodium (DSS, MW 36,000 to 50,000, MP Biomedicals, Solon, Ohio) in drinking water for 8 days; control mice received regular drinking water. During the period when DSS was administered, treated mice were given MC-12 at 5 or 25 mg/kg intraperitoneally (ip) whereas the control group was given saline ip. As above, the mice were weighed and monitored for rectal bleeding or prolapse daily. All mice were euthanized at the end of the study period. Blood samples were collected and colons were dissected and their length was measured. The middle part of colon was fixed in 4% neutralized formalin and the rest was frozen for molecular analyses. Paraffin-embedded tissue sections were stained and scored as above.

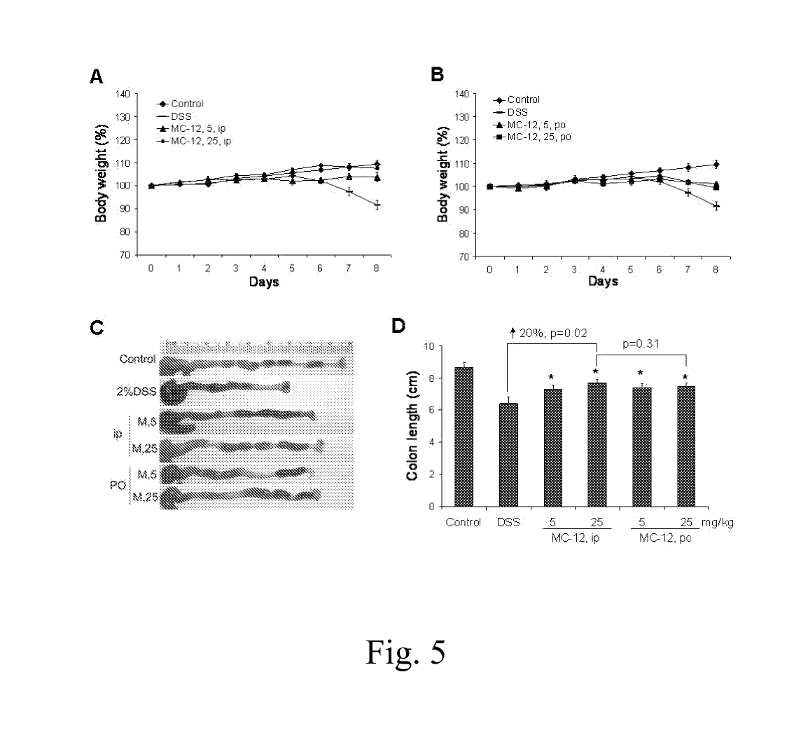

By day 8, mice receiving DSS lost on average 8.4% of their baseline weight (FIG. 5A, B). MC-12 prevented such weight loss in a dose-dependent manner, with those receiving the highest ip dose of MC-12 (500 μg/day), showing 7.7% increase in their weight compared to baseline. MC-12 was more effective in preventing weight loss when given ip compared to po. FIGS. 5C and 5D show changes in the length of the colon of these mice. Compared to control, the colon of DSS-treated mice was shorter by 2.3 cm (p<0.01). Administration of MC-12 prevented this reduction in colon length in a dose-dependent manner, with ip and po administration producing essentially identical results. Macroscopically, shortened colons show wall edema and fewer feces in the lumen.

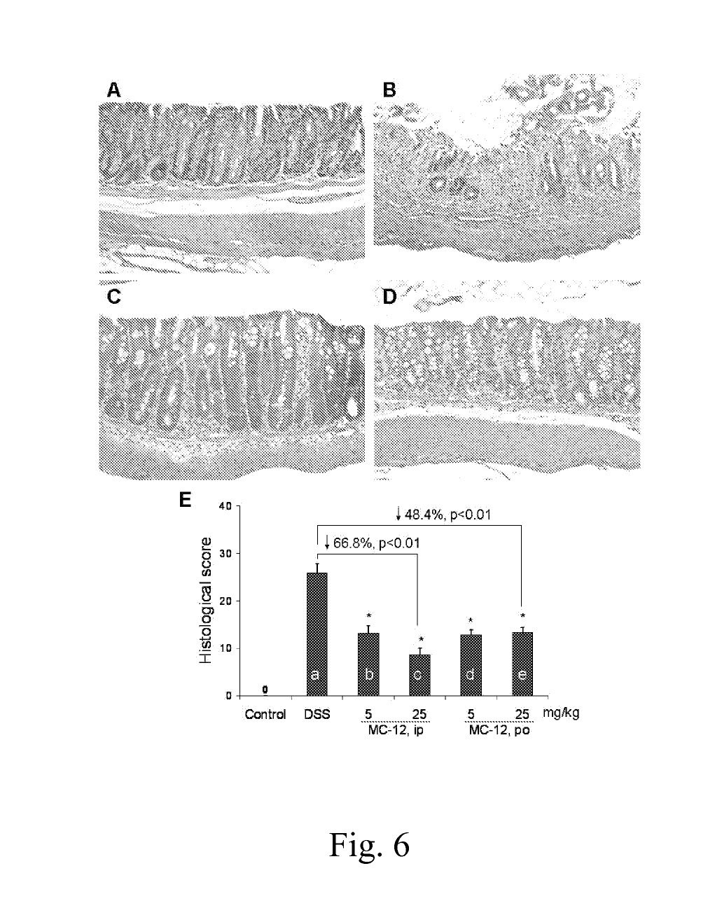

As expected, DSS induced colitis in these mice. The histological sections shown in FIG. 6 demonstrate changes in the colonic mucosa by DSS, including significant inflammation, accumulation of mucus and development of ulcers. The granulocytes present in the mucosa establish the development of acute inflammation. Treatment with MC-12 reduced both the degree of inflammation and the size of goblet cells, essentially restoring the integrity of the mucosa. As shown in FIG. 6, after treatment with DSS the histological colitis score, 0 for controls, became 25.8±2.01 (mean±SEM, for this and all subsequent values). MC-12 reduced the histological colitis score dose-dependently, regardless of its route of administration. IP administration of MC-12 reduced the histological score by 48.9% in 5 mg/kg, ip (p<0.01); 66.8% in 25 mg/kg, ip (p<0.01), compared to DSS control. Orally administered MC-12 was also effective, reducing this histological score by 50.2% at 5 mg/kg, po (p<0.01) and 48.4% at 25 mg/kg, po (p<0.01).

### Example 5

### MC-12 Reduces Experimental Crohn's Disease in Mice

TNBS (2,4,6-trinitro benzene sulfonic acid) induces a form of colitis in SJL/J mice that recapitulates Crohn's disease. The optimal dose of TNBS was determined to be 100 μl of a 2.5% ethanolic solution instilled intracolonically. SJL mice received 2.5% TNBS solution in 50% ethanol by intra-colonic instillation using a 3.5 F catheter, which was inserted 4 cm into the colon under mild ketamine/xylazine anesthesia. Mice received MC-12 25 mg/kg or vehicle ip twice a day for two days. Body weight was monitored daily and mice were euthanized on the third day when blood and colon tissues were collected as described above for further analysis.

The fixed colon was cut into six equal fragments, dehydrated and embedded into paraffin. The tissue sections were stained with hematoxylin and eosin (H&E). The histological score was determined by two pathologists as described, based on the degree (0-3) and extent (0-3) of inflammation, crypt damage (0-4) and the area involved (0-4). The scores of each of the first three parameters were multiplied by the fourth and the sum of these three multiples was the final score (ranging from 0 to 40).

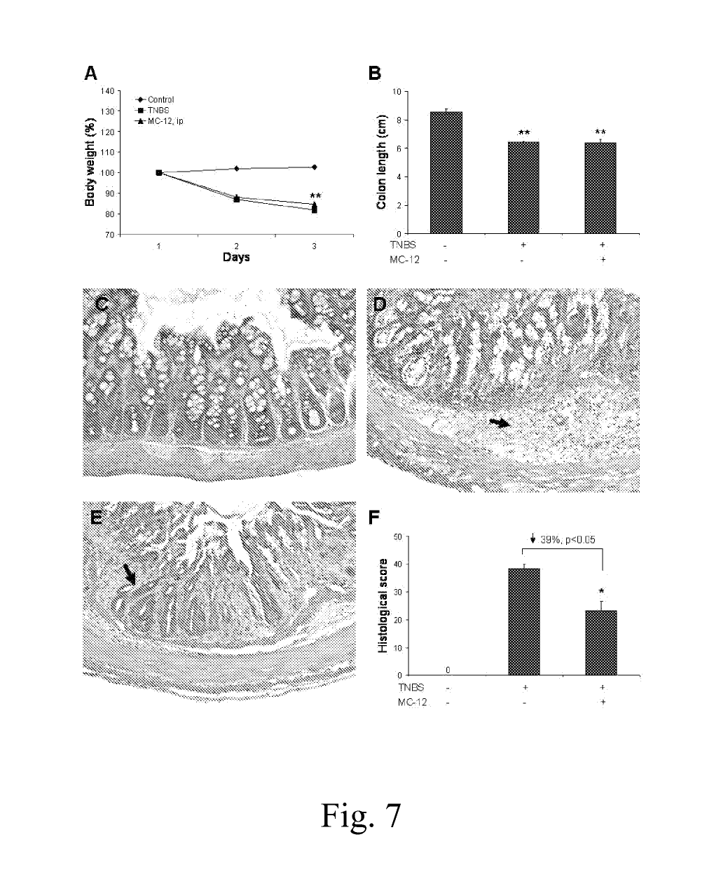

As expected, TNBS induced severe crypt destruction and massive infiltration of inflammatory cells in the entire colonic wall (FIG. 7). This is reflected in a marked increase of the histology score, which reached 38.3±1.7 (0 in control animals). TNBS also reduced the body weight of the animals on day 3 by 18.2% compared to baseline. MC-12 did not prevent weight loss at the dose of 25 mg/kg, reducing it by 15.3%. Similarly, TNBS caused shortening in the length of the colon and MC-12 at 25 mg/kg did not prevent this effect. In contrast, however, MC-12 had a significant anti-inflammatory effect on the colonic mucosa, reducing the histological score by 39.1%, compared to TNBS control, changing it from 38.3±1.7 to 23.3±3.3 (p<0.01).

### Example 6

### MC-12 Reduces Inflammation in Murine Colonic Mucosa Induced by DSS

To assess the effect of MC-12 on the inflammatory changes associated with DSS-induced colitis, myeloperoxidase activity in tissue samples was determined as above.

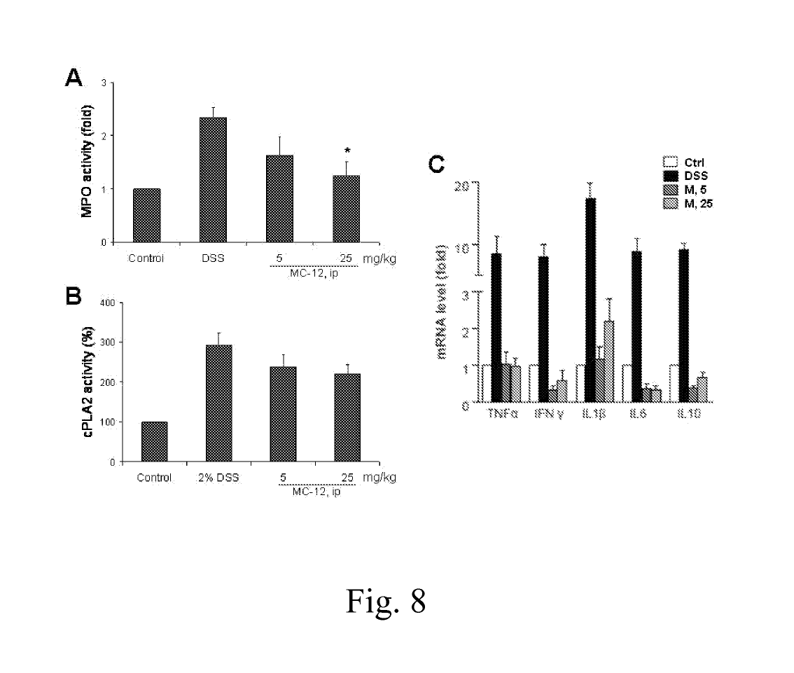

As shown in FIG. 8A, DSS increased MPO activity 2.4-fold compared to normal controls. Mice treated with MC-12 had a significant (p<0.01) and concentration-dependent reduction in MPO activity, which was essentially normalized at the dose of 25 mg/kg.

We also determined the activity of cytosolic phospholipase A₂ (cPLA₂) in colonic tissues from study mice. cPLA₂s are enzymes that release fatty acids from the second carbon group of glycerol. This particular phospholipid specifically recognizes the sn-2 acyl bond of phospholipids and catalytically hydrolyzes the bond releasing arachidonic acid and lysophospholipids, which are then modified into active compounds called eicosanoids by cyclooxygenases. Eicosanoids include prostaglandins and leukotrienes which are categorized as inflammatory mediators.

A cPLA2 assay kit (Cayman, Ann Arbor, Mich.) was used to determine cPLA2 activity following the instructions of the manufacturer. Briefly, a portion of colon tissue was homogenized in cold PBS and centrifuged at 10,000×g for 15 minutes and 10 μl of supernatant from each sample and 5 μl assay buffer were added into a 96-well microplate. The reaction was initialized by adding 200 μl substrate solutions to all wells and incubating for 5 min at room temperature. The fluorescence intensity was measured using a Multiplate Reader (Molecular Devices) at excitation at 485 nm, emission at 530 nm.

As shown in FIG. 8B, DSS increased cPLA₂ activity 2.9-fold compare to controls. Treatment with MC-12 decreased cPLA₂ activity by 18% at 5 mg/kg and 24% at the dose of 25 mg/kg (p<0.05).

### Example 7

### MC-12 Inhibits DSS-Induced Cytokine Increase in Colitis

Expression of inflammatory cytokines TNF-α, IFN-γ, IL-1β, IL-6 and IL-10 in the colon was determined by measuring corresponding mRNA levels using real-time PCR. About 100 mg colon tissue were placed in 1 ml of cold Trizol (Invitrogen Life Technologies, Carlsbad, Calif.), immediately homogenized with a rotor power homogenizer, and RNA was extracted according to the manufacturer's instructions. Total RNA was retrotranscribed with M-MLV reverse transcriptase (Invitrogen Life Technologies, Carlsbad, Calif.) using random primers. Real-time quantitative PCR was performed in a CFD-3200 Opticon detector (BioRad, Hercules, Calif., USA) using QuantiTect SYBR Green PCR Kits (Qiagen, Valencia Calif., USA). The PCR cycling conditions were: 40 cycles of 60 seconds at 94° C., 30 seconds at 51.4° C. and 30 seconds at 72° C. PCR primers (forward and reverse primers) were designed based on published sequences: TNF-α: AGGCTGCCCCGACTACGT (SEQ ID NO:12) and GACTTTCTCCTGGTATGAGATAGCAAA (SEQ ID NO:13); IFN-γ: CAGCAACAGCAAGGCGAAA (SEQ ID NO:14) and CTGGACCT GTGGGTTGTTGAC (SEQ ID NO:15); IL-1β: TCGCTCAGGGTCACAAGAAA (SEQ ID NO:16) and CATCAGAGGCAAGGAGGAAAAC (SEQ ID NO:17); IL-6: ACAAGTCGGAGGCTTAATTACACAT (SEQ ID NO:18) and ATGTGTAATTAAGCCTCCGACTTGT (SEQ ID NO:19); IL-1β: ATGCTGCCTGCTCTTACTGACTG (SEQ ID NO:20) and TTGCCATTGCACAACTCTTTTC (SEQ ID NO:21); β-actin: AGATTACTGCTCTGGCTCCTA (SEQ ID NO:20) and CAAAGAAAGGGTGTAAAACG (SEQ ID NO:21). Relative expression levels of mRNA were normalized to β-actin.

As shown in FIG. 8C, DSS increased the mRNA level of all these cytokines by 9- to 17-fold, compared to controls. Treatment with MC-12 at either dose essentially normalized the mRNA levels of TNF-α and IL-1β, while it reduced those of IFN-γ, IL-6 and IL-10 below baseline (control mice not treated with DSS).

### Example 8

### MC-12 Inhibits the Activation of NF-κB In Vivo and In Vitro

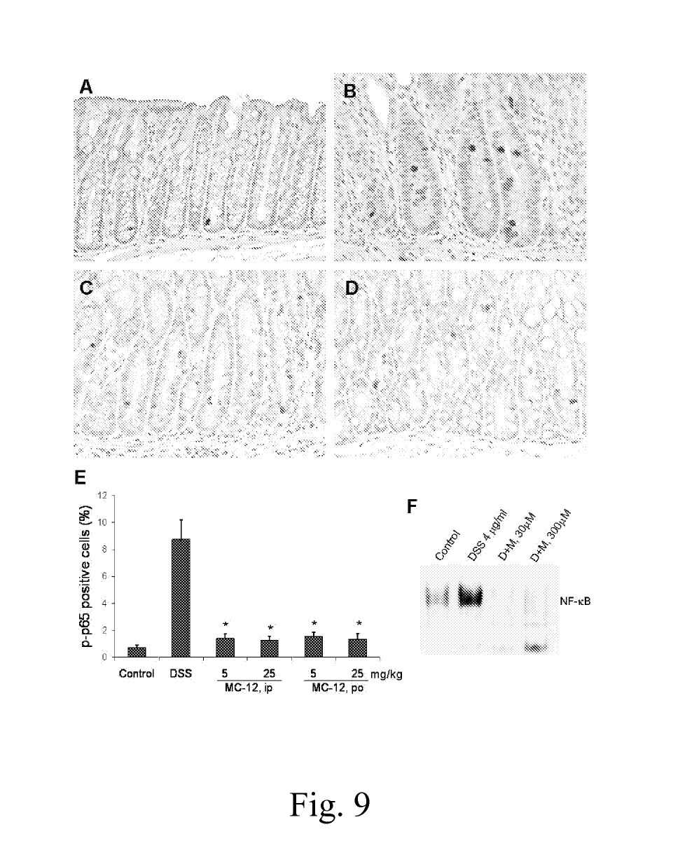

The level of NF-κB activation in the colonic mucosa was determined by immunohistochemistry (FIG. 9). NF-κB is the molecular target of MC-12. Immunohistochemical staining for phospho-NF-κB (activate form of NF-κB) was performed on the colon tissue samples. Briefly, paraffin-embedded sections (4 μm thick) were deparaffinized, rehydrated, and microwave heated for 15 minutes in 0.01 mol/L citric acid buffer (pH 6.0) for antigen retrieval. Then, 3% hydrogen peroxide was applied to block endogenous peroxidase activity. After 30 minutes of blocking with normal serum (Invitrogen, Carlsbad, Calif.), the primary rabbit anti-phospho-NF-κB p65 antibody (Cell Signaling) or corresponding control isotype IgG were applied and incubated overnight at 4° C. Slides were washed thrice with PBS, each for 5 minutes. The biotinylated secondary antibody and the streptavidin-biotin complex were applied, each for 60 minutes incubation and following a thrice-wash at room temperature. After rinsing with PBS, the slides were immersed for 10 minutes in 3,3′-diaminobenzidine (Sigma, St. Louis, Mo.) solution (0.4 mg/mL, with 0.003% hydrogen peroxide), monitored under microscope. The reactions were stopped with distilled water. The slides were counterstained with hematoxylin, dehydrated, and coverslipped. Five photos per section were taken and the positive luminosity was evaluated using PhotoShop program. The result shows the intensity of activate NF-κB in a defined area of colonic mucosa.

There was minimal baseline activation of NF-κB in the colon of control animals. DSS, as expected, activated NF-κB in both colonic crypts and stromal cells. MC-12 markedly decreased this effect of DSS in a clear dose-dependent manner, essentially normalizing it at the highest dose (25 mg/kg). The route of administration did not seem to make a difference with respect to this effect. This inhibitory effect of MC-12 was also detected in cultured NCM 460 cells (normal colon epithelial cells), in which DSS significantly increased NF-κB-DNA binding activity. MC-12 at concentrations of 30 μM and 300 μM essentially eliminated NF-κB activation.

### Example 9

### MC-12 Derivatives with Increased Hydrophobicity have Improved Anti-Inflammatory Properties

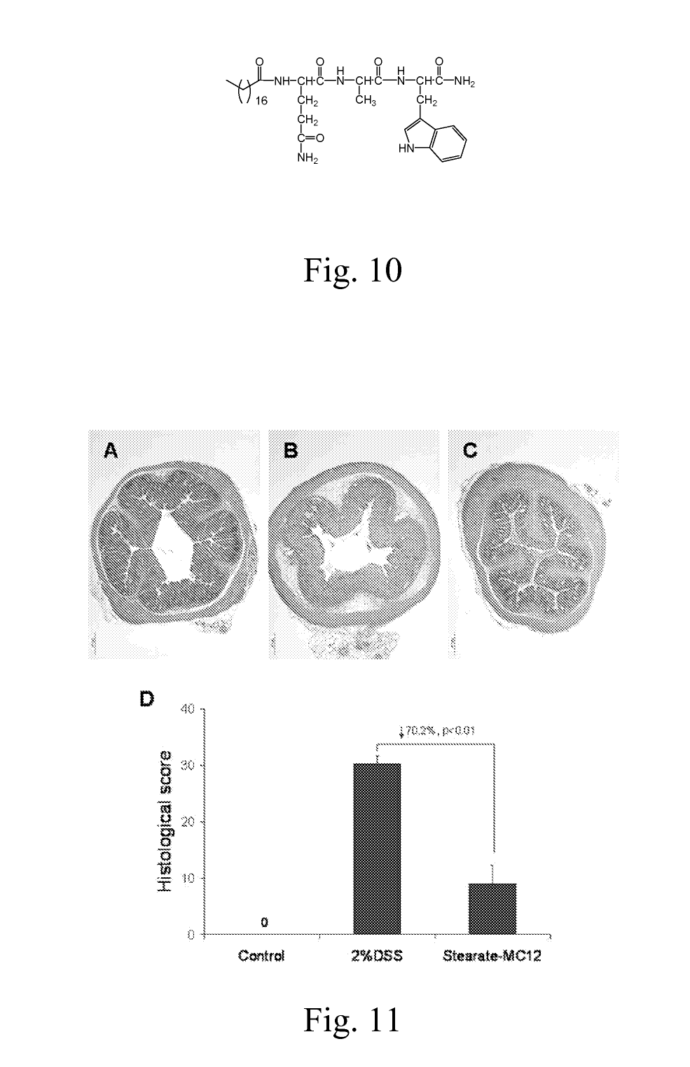

MC-12 was modified by adding it to it stearic acid, generating the stearate-MC12 (StMC12), whose chemical formula is shown in FIG. 10. Addition of the stearic acid moiety increased the lipophilicity of MC12. StMC12 was dissolved in corn oil and administered by oral gavage at a dose of 760 μg/day (equivalent to 500 μg of MC12) daily for 8 days starting on the same day with DSS. Animals were euthanized on day 8 and their colons were evaluated as previously.

As shown in FIG. 11, StMC12 reduced DSS-induced colitis compared to untreated mice that received only DSS. The histological scores of the three groups of mice (8 mice/group) were as follows: untreated mice=0; 2% DSS=30.3±1.35; 2% DSS treated with StMC12=9.0±3.27 (mean±SEM), representing a 70.2% reduction by StMC12 (p=0.0007). Mice treated with StMC12 had no signs of toxicity, tolerating this novel agent well.

### Example 9

### Annexin-1 Peptides Composed of D Amino Acids and Cyclized Annexin-1 Peptides are Anti-Inflammatory Agents for Treating IBDs

The following acetylated peptides, composed of D amino acids, were synthesized and tested for inhibition of NF-kB: 1) Ac-dGln-dALa-dTrp (SEQ ID NO:9); and 2) Ac-dPhe-dLeu-dLys-dGln-dALa-dTrp (SEQ ID NO:10). Both peptides demonstrated effective inhibition of NF-kB and can be used to treat inflammatory bowel diseases. The following acetylated peptide, cyclized through its N- and C-terminals, was synthesized and tested for inhibition of NF-kB: Ac-Phe-Leu-Lys-Gln-Ala-Trp (SEQ ID NO:6). The acetylated cyclized peptide demonstrated effective inhibition of NF-kB and can be used to treat inflammatory bowel diseases.

We claim:

1. A pharmaceutical composition for treatment or prevention of an inflammatory bowel disease which comprises a therapeutically effective amount of an annexin 1 peptide, or a conjugate thereof, comprising Gln-Ala-Trp (SEQ ID NO:1).

2. The pharmaceutical composition of claim 1, wherein the peptide is conjugated to a fatty acid.

3. The pharmaceutical composition of claim 1, wherein the peptide is conjugated to stearic acid.

4. The pharmaceutical composition of claim 1, wherein the peptide is acetylated.

5. The pharmaceutical composition of claim 1, wherein the peptide is cyclic.

6. The pharmaceutical composition of claim 1, wherein the peptide comprises D amino acids.

7. The pharmaceutical composition of claim 1, wherein the peptide consists of D amino acids.

8. The pharmaceutical composition of claim 1, wherein the peptide conjugate is st-Gln-Ala-Trp (SEQ ID NO:11).

9. The pharmaceutical composition of claim 1, wherein the peptide is Gln-Ala-Trp (SEQ ID NO:1) or Ac-Gln-Ala-Trp (SEQ ID NO:2)

10. The pharmaceutical composition of claim 1, wherein the peptide conjugate is Ac-dGln-dAla-dTrp (SEQ ID NO:9) or Ac-dPhe-dLeu-dLys-dGln-dAla-dTrp (SEQ ID NO:10).

11. The pharmaceutical composition of claim 1, wherein the peptide is cyclic Ac-Phe-Leu-Lys-Gln-Ala-Trp (SEQ ID NO:6).

12. The pharmaceutical composition of claim 1, wherein the therapeutically effective amount provides a dose to a human of about 0.01 to about 50 mg/kg.

13. The pharmaceutical composition of claim 1, wherein the therapeutically effective amount provides a dose to a human of about 0.02 to about 25 mg/kg

14. The pharmaceutical composition of claim 1, wherein the therapeutically effective amount provides a dose to a human of about 0.05 to about 2.5 mg/kg.

15. The pharmaceutical composition of claim 1, wherein the inflammatory bowel disease is ulcerative colitis.

16. The pharmaceutical composition of claim 1, wherein the inflammatory bowel disease is Crohn's disease.

17. An annexin 1 peptide conjugate comprising a fatty acid and an annexin-1 peptide comprising Gln-Ala-Trp (SEQ ID NO:1).

18. The annexin 1 peptide conjugate of claim 17, wherein the fatty acid is stearic acid.

19. The annexin 1 peptide conjugate of claim 17, wherein the annexin 1 peptide consists of Gln-Ala-Trp (SEQ ID NO:1).

20. The annexin 1 peptide conjugate of claim 17, wherein the fatty acid is stearic acid and the annexin 1 peptide consists of Gln-Ala-Trp (SEQ ID NO:1).

21. A method of treating or preventing an inflammatory bowel disease in a mammal in need thereof, comprising administering to a mammalian subject a therapeutically effective amount of an annexin 1 peptide, or conjugate thereof, comprising Gln-Ala-Trp (SEQ ID NO:1).

22. The method of claim 21, wherein the peptide is conjugated to a fatty acid.

23. The method of claim 21, wherein the peptide is conjugated to stearic acid.

24. The method of claim 21, wherein the peptide is acetylated.

25. The method of claim 21, wherein the peptide is cyclic.

26. The method of claim 21, wherein the peptide comprises D amino acids.

27. The method of claim 21, wherein the peptide consists of D amino acids.

28. The method of claim 21, wherein the peptide conjugate is st-Gln-Ala-Trp (SEQ ID NO:11).

29. The method of claim 21, wherein the peptide is Gln-Ala-Trp (SEQ ID NO:1) or Ac-Gln-Ala-Trp (SEQ ID NO:2).

30. The method of claim 21, wherein the peptide is Ac-dGln-dAla-dTrp (SEQ ID NO:9) or Ac-dPhe-dLeu-dLys-dGln-dAla-dTrp (SEQ ID NO:10).

31. The method of claim 21, wherein the peptide is cyclic Ac-Phe-Leu-Lys-Gln-Ala-Trp (SEQ ID NO:6).

32. The method of claim 21, wherein the peptide is administered intraperitonealy.

33. The method of claim 21, wherein the peptide is administered orally.

34. The method of claim 33, wherein the peptide is administered in corn oil.

35. The method of claim 21, wherein the inflammatory bowel disease is ulcerative colitis.

36. The method of claim 21, wherein the inflammatory bowel disease is Crohn's disease.

37. The method of claim 21, wherein the mammal is a human.

38. The method of claim 37, wherein the peptide is administered in an amount from about 0.01 to about 50 mg/kg.

39. The method of claim 37, wherein the peptide is administered in an amount from about 0.02 to about 25 mg/kg.

40. The method of claim 37, wherein the peptide is administered in an amount from about 0.05 to about 2.5 mg/kg.
